(19) United States
(12) Patent Application Publication
(10) Pub. No.: US 2020/0390463 A1
(43) Pub. Date: Dec. 17, 2020

(54) DUAL-BLADE TENDON CUTTING APPARATUS AND CARTRIDGE FOR MULTIPLE APPARATUSES

(71) Applicant: Quadvantage Technology, Inc., Raleigh, NC (US)

(72) Inventor: Paul Leach Burroughs, III, Raleigh, NC (US)

(21) Appl. No.: 16/900,315

(22) Filed: Jun. 12, 2020

Related U.S. Application Data
(60) Provisional application No. 62/861,623, filed on Jun. 14, 2019.

(51) Int. Cl. A61B 17/3215; A61B 17/3213; A61B 17/3205

(52) U.S. Cl. CPC A61B 17/3215; A61B 2017/0023; A61B 17/3205; A61B 17/3213

(57) ABSTRACT

The present disclosure provides a dual-blade tendon cutting apparatus and cartridge for multiple apparatuses. Exemplary aspects of the present disclosure relate to a cutting implement that comprises two parallel blades that are spaced apart from one another by a predefined distance. The distance between the blades determines a lateral size of a graft being taken from the quadriceps tendon. To simplify matters for the surgeon, a cartridge is provided with three different blade sets. Much like a razor that has a disposable head, the cutting implement may attach to a desired blade set and be used.

### PRIORITY CLAIM

The present application claims priority to U.S. Provisional Patent Application Ser. No. 62/861,623 filed on Jun. 14, 2019 and entitled “DUAL-BLADE TENDON CUTTING APPARATUS AND CARTRIDGE FOR MULTIPLE APPARATUSES,” the contents of which is incorporated herein by reference in its entirety.

### FIELD OF THE DISCLOSURE

The present disclosure relates to a dual-blade cutting apparatus for cutting tendons and, more particularly, for cutting a quadriceps tendon and a cartridge system for holding multiples ones of the dual-blade cutting apparatus.

### BACKGROUND

Most people can go through the majority of their life without ever caring or knowing how complicated a structure the knee that helps them walk is. However, the knee remains a fragile mechanical structure that is readily susceptible to damage. While medical advances have made repairing the knee possible, repair of certain types of injuries results in other long term effects. To assist the reader in appreciating the elegance of the present disclosure, FIG. 1 is provided with a brief explanation of the components of the knee.

For the purposes of the present disclosure, and as illustrated, the knee may be composed of the quadriceps muscles 100, the femur 102, the articular cartilage 104, the lateral condyle 106, the posterior cruciate ligament 108, the anterior cruciate ligament (ACL) 110, the lateral collateral ligament 112, the fibula 114, the tibia 116, the patellar tendon 118, the meniscus 120, the medial collateral ligament 122, the patella 124 (shown slightly displaced to the side—it normally rests in the center of the knee), and the quadriceps tendon 126. Of particular interest for the purposes of the present disclosure is the ACL 110 and what is done to repair the ACL 110.

ACL tears are common in athletes and are usually season-ending injuries. The ACL 110 cannot heal—it must be surgically reconstructed. The reconstruction requires replacement tissue. The most common tissue used is a central slip of the patient's own patellar tendon 118. In practice, the patellar tendon 118 has proven to be generally effective, but the size of the graft that can be used is limited to the size of the patient's own patellar tendon 118. As a rule of thumb, only a third of the patellar tendon 118 may be harvested as a graft. Thus, a doctor will measure the width of the patellar tendon 118, divide by three, and take the middle third of the patellar tendon 118. Such harvested grafts are rarely more than ten millimeters (10 mm) wide and may be smaller. Taking this tissue from a person's patellar tendon 118 also causes significant pain and discomfort in the post-operative healing period, which may last up to a year, and up to twenty (20) percent of these patients are left with chronic anterior knee pain.

Some doctors recommend and use other graft sources, such as cadaver grafts, but cadaver grafts have a higher failure rate. Additionally, there is a non-zero chance of disease transmission or rejection by the patient's immune system. As a final drawback, cadaver grafts are usually quite expensive and may not be covered by some insurance companies.

Other doctors use hamstring tendons (e.g., the distal semitendinosus tendon) because the scar created during harvesting is relatively small and there is less pain during the rehabilitation, but again, the hamstring tendon has its own collection of disadvantages. The disadvantages include the fact that once the graft is taken, a patient's hamstring will never recover to its previous strength. Further, all hamstring reconstructions stretch and are looser than the original ACL 110. This loosening is particularly problematic in younger female athletes.

Another alternative graft source is the quadriceps tendon 126. The quadriceps tendon 126 is larger and stronger than either the patellar tendon 118 or the hamstring tendon. The quadriceps tendon 126 is likewise stiffer and less prone to stretching or plastic deformation. However, the qualities that make the quadriceps tendon 126 attractive also contribute to the difficulty in harvesting a graft from the quadriceps tendon 126. Existing surgical implements require a large incision up the longitudinal axis of the femur 102 on the front or ventral/anterior side of the thigh to cut down to the level of the quadriceps tendon 126, resulting in a large post-operative scar. Additionally, the quadriceps tendon 126 has a consistency similar to the proverbial shoe leather, making it difficult to cut. However, an ACL 110 repaired with grafts from the quadriceps tendon 126 generally result in almost no anterior knee pain postoperatively over the short or long term and patients recover quicker.

U.S. Pat. Nos. 8,894,672; 8,894,675; 8,894,676; 9,044,260; 9,107,700; and 9,474,535 provide a number of devices designed to create a graft from the quadriceps tendon 126 as well as a number of secondary cutting implements to trim the distal end of the graft. While these devices perform admirably, there is a desire to afford a surgeon more flexibility in approaching tendon harvesting.

### SUMMARY

The present disclosure provides a dual-blade tendon cutting apparatus and cartridge for multiple apparatuses. Exemplary aspects of the present disclosure relate to a cutting implement that comprises two parallel blades that are spaced apart from one another by a predefined distance. The distance between the blades determines a lateral size of a graft being taken from the quadriceps tendon. To simplify matters for the surgeon, a cartridge is provided with three different blade sets. Much like a razor that has a disposable head, the cutting implement may attach to a desired blade set and be used.

In this regard, in one aspect, a cartridge is disclosed. The cartridge includes a generally rectangular housing with a plurality of bays therein to hold a plurality of dual-blade cutting attachments.

In another aspect, a surgical instrument is disclosed. The surgical instrument includes a plurality of dual-blade cutting attachments. The surgical instrument also includes a cartridge. The cartridge includes a generally rectangular housing with a plurality of bays therein to hold the plurality of dual-blade cutting attachments. The surgical instrument also includes a handle configured to snap fit with any one of the plurality of dual-blade cutting attachments.

Those skilled in the art will appreciate the scope of the disclosure and realize additional aspects thereof after reading the following detailed description in association with the accompanying drawings.

### BRIEF DESCRIPTION OF THE DRAWINGS

The accompanying drawings incorporated in and forming a part of this specification illustrate several aspects of the disclosure, and together with the description serve to explain the principles of the disclosure.

FIG. 1 illustrates a conventional knee;

FIG. 2A illustrates a perspective top view of a dual-blade cutting apparatus according to an exemplary aspect of the present disclosure;

FIG. 2B illustrates a side elevational view of the dual-blade cutting apparatus of FIG. 2A;

FIG. 2C is a top plan view of the dual-blade cutting apparatus of FIG. 2A;

FIG. 2D is a front side elevational view of the dual-blade cutting apparatus of FIG. 2A;

FIG. 3A illustrates a top plan view of a cartridge holding a plurality of dual-blade attachments suitable for use with a handle;

FIG. 3B illustrates a side elevational view of the cartridge of FIG. 3A taken along line 3B-3B of FIG. 3A;

FIG. 3C illustrates a longitudinal side elevational view of the cartridge of FIG. 3A taken along line 3C-3C of FIG. 3A;

FIG. 3D illustrates a perspective view of the cartridge of FIG. 3A with a plurality of differently-sized dual-blade attachments ready for use;

FIG. 4A illustrates a top front perspective view of a dual-blade attachment removed from the cartridge of FIG. 3A;

FIG. 4B illustrates a top plan view of the dual-blade attachment of FIG. 4A;

FIG. 4C illustrates a side elevational view of the dual-blade attachment of FIG. 4A;

FIG. 4D illustrates a rear side elevational view of the dual-blade attachment of FIG. 4A; and

FIG. 5 illustrates a perspective view of a dual-blade attachment secured to a handle proximate a cartridge holding a plurality of dual-blade attachments.

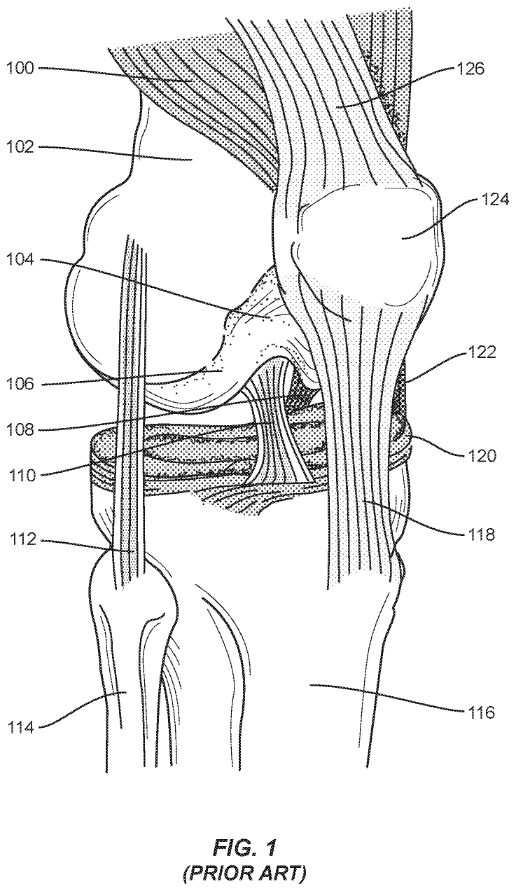

### DETAILED DESCRIPTION

The embodiments set forth below represent the necessary information to enable those skilled in the art to practice the disclosure and illustrate the best mode of practicing the disclosure. Upon reading the following description in light of the accompanying drawings, those skilled in the art will understand the concepts of the disclosure and will recognize applications of these concepts not particularly addressed herein. It should be understood that these concepts and applications fall within the scope of the disclosure and the accompanying claims.

The present disclosure provides a dual-blade tendon cutting apparatus and cartridge for multiple apparatuses. Exemplary aspects of the present disclosure relate to a cutting implement that comprises two parallel blades that are spaced apart from one another by a predefined distance. The distance between the blades determines a lateral size of a graft being taken from the quadriceps tendon. To simplify matters for the surgeon, a cartridge is provided with three different blade sets. Much like a razor that has a disposable head, the cutting implement may attach to a desired blade set and be used.

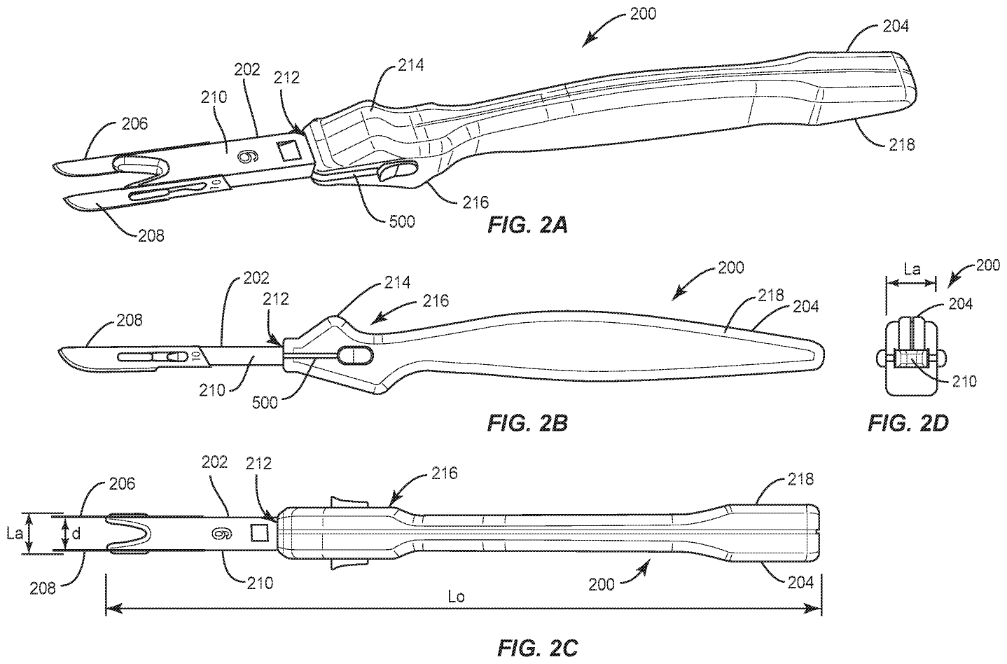

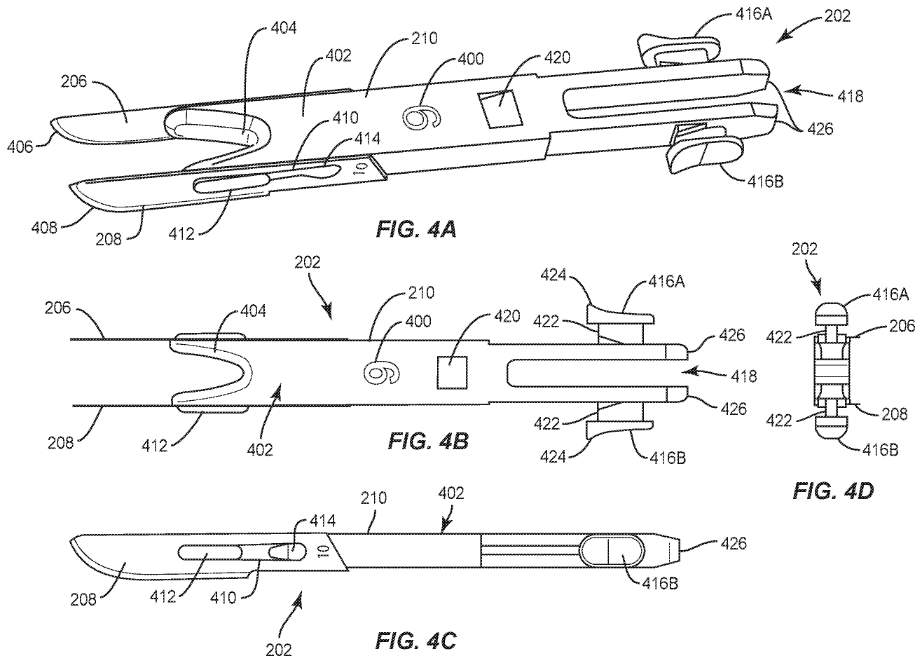

FIGS. 2A-2D illustrate a variety of views of a surgical instrument 200 that has a dual-blade cutting attachment 202 secured to a handle 204 through a snap fit arrangement. The dual-blade cutting attachment 202 has a first blade 206 in parallel with a second blade 208 along a longitudinal axis Lo, and spaced apart from one another by a distance d along a lateral axis La. The distance d corresponds to a desired dimension of a graft being harvested by the surgical instrument 200. Thus, for example, if there are nine millimeters (9 mm) between the first blade 206 and the second blade 208, a graft 9 mm wide may be harvested. The blades 206, 208 may be a metal such as surgical grade steel, although diamond, flint, or obsidian may also be used as needed or desired. The blades 206, 208 are secured to abridge 210. The bridge 210 may be generally planar or rectilinear with a U-shaped front portion. More detail about the dual-blade attachment 202 is provided below with reference to FIGS. 4A-4D.

The handle 204 includes a channel 212 and beveled edges to allow the dual-blade cutting attachment 202 to snap fit therein. The handle 204 is contoured to provide an easy grip surface and may be made from plastic or the like. In particular, the handle 204 may include a first front ridge 214 where pressure may be applied such as by a surgeon's thumb. Likewise, the handle 204 may be generally I-shaped with a wider front section 216 and rear section 218 (best seen in FIG. 2C) to prevent or reduce the chance of a surgeon's hand slipping in the longitudinal direction Lo.

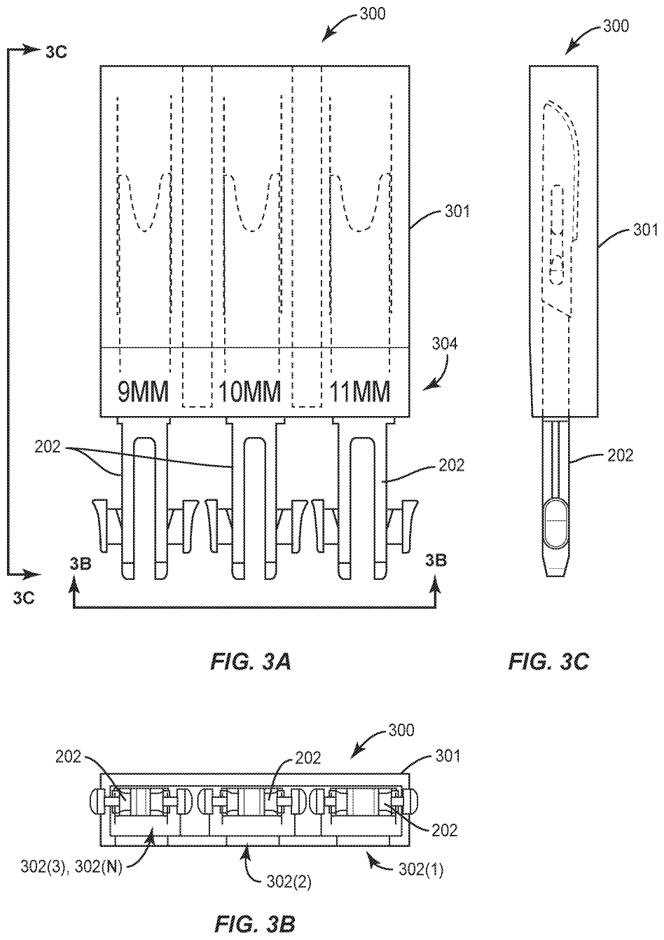

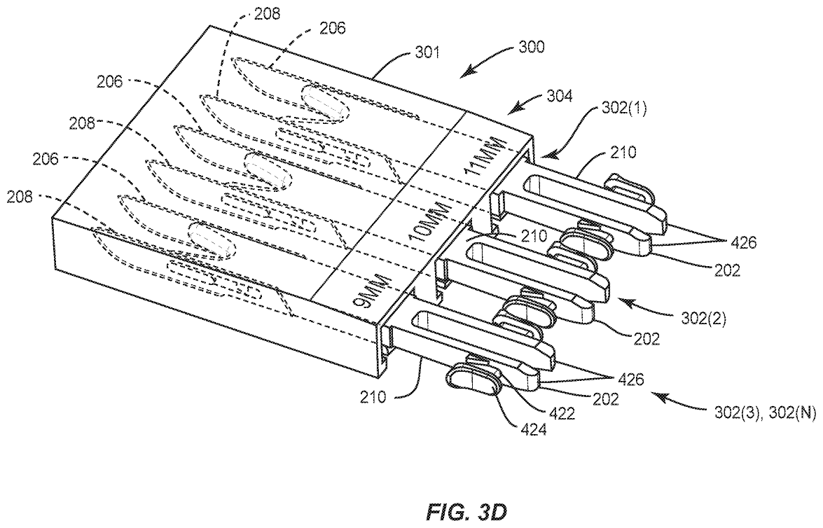

Because the size of the graft may vary depending on the person, it may be appropriate to use differently-sized dual-blade cutting attachments 202. To this end, exemplary aspects of the present disclosure allow a plurality of differently-sized dual-blade cutting attachments to be placed into respective bays of a single cartridge 300 as illustrated in FIGS. 3A-3D. In particular, the cartridge 300 may include a housing 301 which may be made of a clear or translucent plastic material and may be generally rectangular or rectilinear and may include a plurality of bays 302(1)-302(N), where as illustrated N is three (3). In the example illustrated, widths of nine millimeters (9 mm), 10 mm, and 11 mm are provided for the bays 302(3)-302(1), respectively, although it should be appreciated that other widths may be provided if needed or desired. Likewise, it is possible to provide multiple dual-blade cutting attachments 202 at the same width. The housing 301 is sized so that the blades 206, 208 of the dual-blade cutting attachment 202 are within the housing of the cartridge while a portion of the bridge 210 is exposed. The housing 301 may include external indicia 304 that provide a visual indication of the distance d for the dual-blade cutting attachment 202 within a given bay 302. The indicia 304 may be raised lettering or cut (e.g., through laser etching) into the housing 301.

The dual-blade cutting attachment 202 is further illustrated in FIGS. 4A-4D. In particular, the bridge 210 may include indicia 400 on an upper surface 402 that indicates the distance d between the blades 206, 208. The U-shaped portion 404 may be spaced from terminal ends 406, 408 of the blades 206, 208. The blades 206, 208 may include channels 410 that snap fit over protuberances 412, 414 of the bridge 210. The bridge 210 may further include two wing-like structures 416A, 416B that bend or compress inwardly as the handle 204 is slid over the handle end 418 of the bridge 210. A recess 420 may cooperate with a protuberance (not shown) in the housing 301 (e.g., in the bays 302) to hold the dual-blade cutting attachment 202 within a bay 302 until drawn out. The wing-like structures 416A, 418 include a narrow portion 422 that fits within a channel on the handle 204 and a wide portion 424 that may be sized and contoured to fit the tip of a finger. Thus, for example, a user may use a thumb and forefinger pinching together to squeeze the wing-like structures 416A, 416B together as the narrow portion 422 is slid into the channel of the handle 204 to insert the bridge 210 into the handle 204. Once forward motion is arrested (either by the protuberance hitting the recess 420 or by terminal ends 426 hitting the end of the channel 212), the user may release the wing-like structures 416A, 416B to create a snap-fit between the bridge 210 and the handle 204.

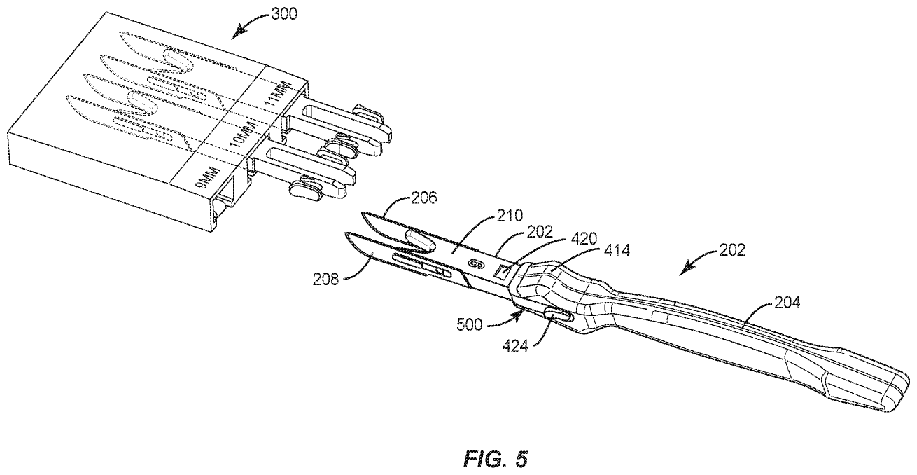

FIG. 5 illustrates the handle 204 being used to capture and extract a dual-blade cutting attachment 202 from the cartridge 300. In use, the handle 204 is grasped in one hand, and the housing 301 held in the other, and the narrow portion 422 of the bridge 210 is threaded into a channel 500 on the side of the handle 204 as the terminal ends 426 are fed into the channel 212 of the handle 204. Once threaded, the user may pinch the wing-shaped structures 416A, 416B until the dual-blade cutting attachment 202 is seated in the handle 204, then the handle 204 is pulled away from the housing 301, drawing the blades 206, 208 out of the bay 302. The surgical instrument 200 is then ready for use in harvesting a tendon.

Those skilled in the art will recognize improvements and modifications to the embodiments of the present disclosure. All such improvements and modifications are considered within the scope of the concepts disclosed herein and the claims that follow.

What is claimed is:

1. A cartridge comprising:
a generally rectangular housing with a plurality of bays therein to hold a plurality of dual-blade cutting attachments.

2. The cartridge of claim 1, further comprising the plurality of dual-blade cutting attachments.

3. The cartridge of claim 2, wherein at least one of the plurality of dual-blade cutting attachments comprises a pair of wing-like structures for snap fit engagement with a handle.

4. The cartridge of claim 2, wherein each of the plurality of dual-blade cutting attachments is a different size relative to other ones of the plurality of dual-blade cutting attachments.

5. The cartridge of claim 4, wherein one is 9 millimeters (mm), one is 10 mm, and one is 11 mm.

6. A surgical instrument comprising:
a plurality of dual-blade cutting attachments;
a cartridge comprising:
a generally rectangular housing with a plurality of bays therein to hold the plurality of dual-blade cutting attachments; and
a handle configured to snap fit with any one of the plurality of dual-blade cutting attachments.

7. The surgical instrument of claim 6, wherein the handle is generally I-shaped.

8. The surgical instrument of claim 6, wherein the handle includes a front ridge.

9. The surgical instrument of claim 6, wherein the handle includes a channel configured to receive any one of the plurality of dual-blade cutting attachments.

10. The surgical instrument of claim 6, wherein the handle includes a channel configured to receive a narrow portion of a wing-like structure.

11. The surgical instrument of claim 6, wherein each of the plurality of dual-blade cutting attachments comprises a bridge.

12. The surgical instrument of claim 6, wherein each of the plurality of dual-blade cutting attachments comprises two blades parallel to one another along a longitudinal axis.

13. The surgical instrument of claim 12, wherein the two blades are formed from surgical grade steel.
